(19) United States
(12) Patent Application Publication
(10) Pub. No.: US 2002/0133096 A1
(43) Pub. Date: Sep. 19, 2002

(54) Distance measuring device

(75) Inventors: Masataka Toda, Toyota-shi (JP); Koji Kuno, Nagoya-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AISIN SEIKI KABUSHIKI KAISHA, Kariya-shi (JP)

(21) Appl. No.: 10/095,032

(22) Filed: Mar. 12, 2002

(30) Foreign Application Priority Data
Mar. 12, 2001 (JP) 2001-068629
Mar. 19, 2001 (JP) 2001-078572

(51) Int. Cl. A61B 5/103

(52) U.S. Cl. 600/590; 433/29

(57) ABSTRACT

A distance measuring device includes an emitting optical fiber 1 for emitting a light beam from its distal end to an object so that the light beam may be reflected on the object, a plurality of side-by-side arranged receiving optical fibers 21-27 for receiving the reflected beam at each distal end a holder 3 holding therein the emitting fiber 1 and the receiving fibers 21-27, an actuator for moving the distal ends of the respective emitting and receiving optical fibers concurrently in a same direction and an analyzer for determining a distance to the object on the basis of a signal derived from the light beam received at the distal end of the receiving optical fibers 21-27. Driving the actuator brings in concurrent movements of the distal ends of the respective emitting and receiving optical fibers in a same direction, which increase the irradiation range of the distance measuring device.

### CROSS-REFERENCE TO RELATED APPLICATIONS

The present application is based on and claims priority under 35 U.S.C. § 119 with respect to Japanese Patent Application Nos. 2001-068629 and 2001-078572 which were filed on Mar. 12, 2001 (13th Year of Heisei) and Mar. 19, 2001 (13th Year of Heisei), respectively, the entire contents of which are incorporated herein by reference.

### BACKGROUND OF THE INVENTION

### FIELD OF THE INVENTION

The present invention is generally directed to a distance measuring device such as a probe for measuring the depth of a periodontal pocket.

Japanese Patent No. 3019857 issued on Jan. 7, 2000 discloses a distance measuring device or a periodontal pocket depth measuring device which includes emitting means for emitting a light beam from its distal end to an object so that the light beam may be reflected on the object; receiving means for receiving the reflected beam at its distal end; a holder holding therein the emitting means and the receiving means; and analyzing means for determining a distance to the object on the basis of a signal derived from the light beam received at the distal end of the receiving means.

This periodontal pocket depth measuring device is capable of measuring the periodontal pocket depth of a patient without having to contact the device with the bottom of the periodontal pocket, which makes it possible to free the patient from pain resulting from contact of a conventional probe with the bottom of the periodontal pocket.

The above-mentioned periodontal depth measuring device, though it has advantages similar to those disclosed in U.S. Pat. No. 5,897,509, is has insufficient measurement precision.

### SUMMARY OF THE INVENTION

Accordingly in order to meet the above need, a first aspect of the present invention provides a distance measuring device which comprises emitting means for emitting a light beam from its distal end to an object so that the light beam may be reflected on the object; receiving means for receiving the reflected beam at its distal end; a holder holding the emitting means and the receiving means; an actuator for moving the distal ends of the respective emitting means and receiving means concurrently and in a same direction; and analyzing means for determining a distance from the emitter means to the object on the basis of a signal derived from the light beam received at the distal end of the receiving means.

A second aspect of the present invention is to provide a distance measuring device whose gist is to modify the structure of the first aspect such that the actuator is selected from one of a piezoelectric element, a bimetal element, a magentostrictor, and an electromagnetic actuator.

A third aspect of the present invention is to provide a distance measuring device whose gist is to modify the structure of the first aspect such that the receiving means is in the form of a plurality of side-by-side arranged optical fibers.

A fourth aspect of the present invention is to provide a distances measuring device whose gist is to modify the structure of the third aspect such that the actuator is selected from one of a piezoelectric element, a bimetal element, a magentostrictor, and an electromagnetic actuator.

A fifth aspect of the present invention is to provide a distance measuring device whose gist as to modify the structure of the first aspect such that the object is a bottom of a periodontal pocket.

A sixth aspect of the present invention is to provide a distance measuring device whose gist is to modify the structure of the first aspect such that (a) the object is a bottom of a periodontal pocket, and (b) the actuator is formed into a sheet configuration whose thickness is directed to a teeth alignment direction.

### BRIEF DESCRIPTION OF THE DRAWINGS

The above and other objects, features and advantages of the present invention will be more apparent and more readily appreciated from the following detailed description of preferred exemplary embodiments of the present invention, taken in connection with the accompanying drawings, in which:

FIG. 1 illustrates an overall structure of a periodontal pocket depth measuring device as a first embodiment of the present invention;

FIG. 2 is an enlarged cross-sectional view of a probe of the device shown in FIG. 1;

FIG. 3 is a cross-sectional view taken along line W3-W3 in FIG. 2;

FIG. 4 is a cross-sectional view taken along line W4-W4 in FIG. 2;

FIG. 5 illustrates a condition under which an actuator bends a distal end of a fiber array;

FIG. 6 is a graph which represents a relationship between a distance and a light amount received at each receiving optical fiber, the distance being measured from a distal end of the probe;

FIG. 7 illustrates an inside structure of the probe of the device shown in FIG. 1;

FIG. 8 illustrates how the distal end of the probe is inserted into the periodontal pocket;

FIG. 9 illustrates how the distal end of the probe is moved along a teeth alignment;

FIG. 10 illustrates how the periodontal pocket depth is measured with the distal end of the probe inserted into the periodontal pocket;

FIG. 11 illustrates an on-screen graphic indication of the distance to the bottom of the periodontal pocket;

FIG. 12 illustrates another on-screen graphic indication of the distance to the bottom of the periodontal pocket;

FIG. 13 illustrates an overall structure of a periodontal pocket depth measuring device as a second embodiment of the present invention;

FIG. 14 is an enlarged cross-sectional view of a probe of the device shown in FIG. 13;

FIG. 15 is a cross-sectional view taken along line W3-W3 in FIG. 14; and

FIG. 16 illustrates how the optical fiber transmits the light beam.

### DETAILED DESCRIPTION OF THE PREFERRED EMBODIMENT

Hereinafter, embodiments of the present invention will be described in great detail with reference to the attached drawings.

[First Embodiment]

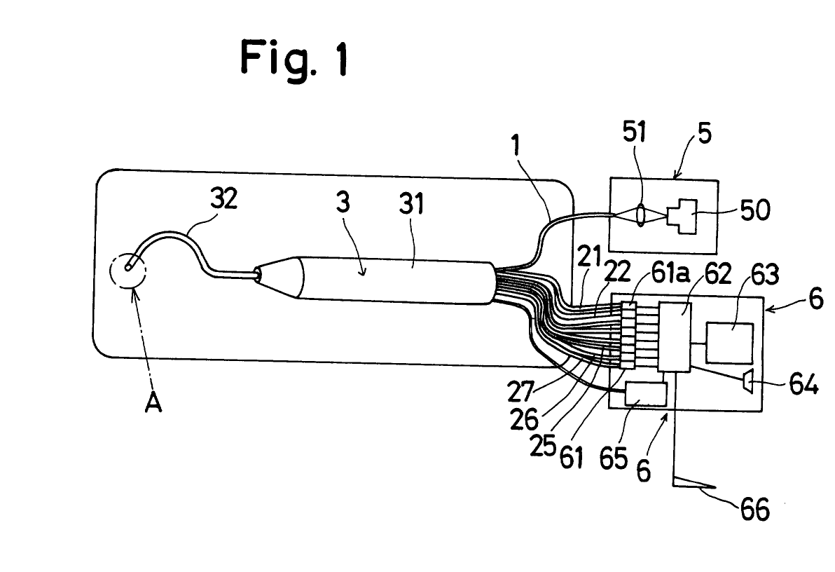

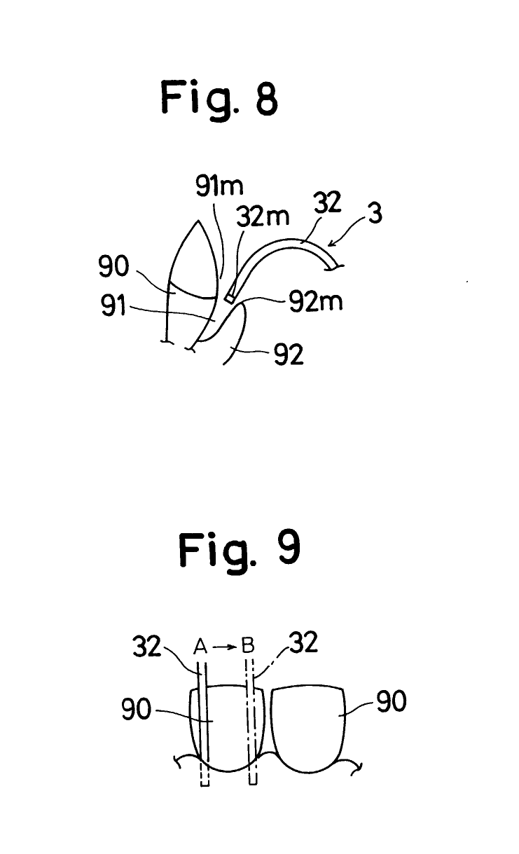

Referring first to FIGS. 1 to 12 inclusive, there is illustrated a periodontal pocket depth measuring device as an embodiment of a distance measuring device in accordance with a first embodiment of the present invention. As illustrated in FIGS. 1 to 3 inclusive, the periodontal pocket depth measuring device is a unit which includes a single emitting optical fiber 1 as an emitting means which emits a light beam from its distal end to a bottom of periodontal pocket 91 (FIG. 8) so that the light beam may be reflected on the bottom of the periodontal pocket 91. A plurality of receiving optical fibers 21-27, as receiving means, receive at their distal ends the reflected light beam. A holder 3 holds therein the emitting optical fiber 1 and the receiving optical fibers 21-27. The holder 3 includes a larger diameter probe cover 31 and a smaller diameter elastic probe 32 which extends therefrom. The probe 32 has a distal end which is indicated as ‘A’.

The periodontal pocket depth measuring device includes a light source unit 5 and a control unit 6 which are placed at input and output sides thereof, respectively. The light source unit 5 has light source 50 which emits a laser beam and a focusing lens 51 to collect the emitted laser beam to input into a proximate or input end of the emitting optical fiber 1. The control unit 6 has a converting portion 61 for converting light signals received at the respective receiving optical fibers 21-27 to electric signals, respectively, a processing portion 62 which determines a distance to the bottom of the periodontal pocket 91 by processing the electric signals obtained at the converting portion 62, a data indicating portion 63, as an indicating means, which indicates the periodontal pocket depth in visual mode based on the signal issued from the signal processing portion 62, a loud speaker 64, as data announcing means, which indicates the periodontal pocket depth in auditory mode based an the signal issued from the signal processing portion 62, a scan control portion 65 which controls behavior of a minute actuator as will be detailed later, and a main switch 66. The converting portion 61 is configured by a plurality of photo-diodes 61a which are optically coupled to the proximate ends of the receiving optical fibers 21-27, respectively. The plurality of the receiving optical fibers 21-27 are in an array. Similarly, the photo-diodes 61a are in an array. The loud speaker 64 issues different sounds depending on the depth of the periodontal pocket.

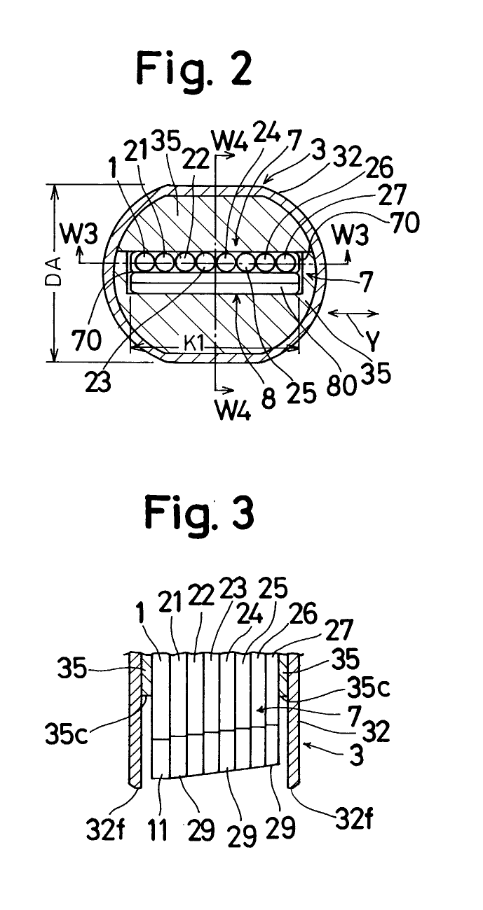

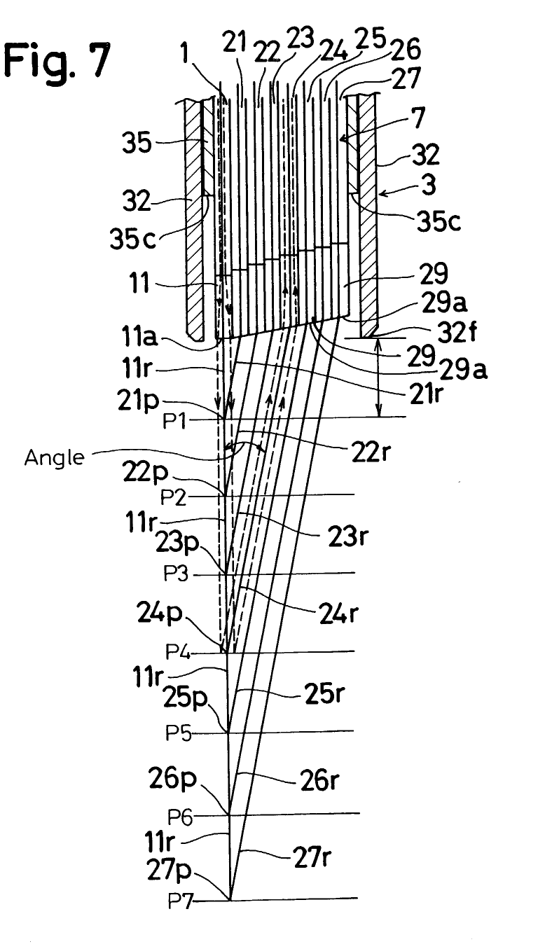

As can be seen from FIG. 2, the single emitting optical fiber 1 and the plurality of receiving optical fibers 21-27 constitute a fiber array 7 with a soldering agent 70 provided between adjacent fibers. The fiber array 7 is placed in the probe 32 which is of a substantial oval cross section. As shown in FIG. 3, the distal end of the emitting optical fiber 1 is covered with a lens 11 for the prevention of light diversion or spread of the emitted light beam. Each of the distal ends of the receiving optical fibers 21-27 is, likewise, covered with a wedge lens 29 which enables each receiving fiber to act as light receiving means. The wedge lens 29 makes the light receiving angle of the corresponding receiving optical fiber small and establishes an intersection between a light emission axis 11r (FIG. 7) and each of light receiving axes 21r/22r/23r/24r/25r/26r/27r of the receiving optical fiber 21/22/23/24/25/26/27. Each of the lens 1 and the wedge lens 29 is in the form of GRIN lens which is of higher refraction rate distribution for enhancing light collection. The lens 11 is adhered to the emitting optical fiber 1 by means of fusion bonding. Likewise, the wedge lenses 29 are adhered to the respective receiving optical fibers 21-27 by means of fusion bonding. As can be seen from FIG. 7, a distal end of each wedge lens 29 is made inclined or slant for making an inclining angle between the light emitting axis 11r and each of the light receiving axis 21r/22r/23r/24r/25r/26r/27r of the receiving optical fiber 21/22/23/24/25/26/27.

As can be seen from FIGS. 2, 4, and 5, a minute actuator 8 is placed close to or next to the fiber array 7 such that the actuator 8 extends along the arranging direction of the fibers as best shown in FIG. 2. In this embodiment the actuator 8 is in the form of bimorph type piezoelectric element which is made up of two layered piezoelectric substances 80, but it could instead be formed of a bimetal element, a magentostrictor or an electromagnetic actuator. Each of the substances 80 has an electrode (not shown) which may be applied with voltage. Upon receipt of a voltage, one of the layered sheet piezoelectric substances 80 expands in its lengthwise direction, which causes the actuator 8 to bend with a snap action like a bimetal element. The resultant bending degree of the actuator 8 will increase more if the other piezoelectric substance 80 is designed to shrink in its lengthwise direction upon receipt of voltage. As best shown in FIG. 2, the width K1 of the actuator 8 is made identical with the width of the fiber array 7 for making the snap action or bending movement effective.

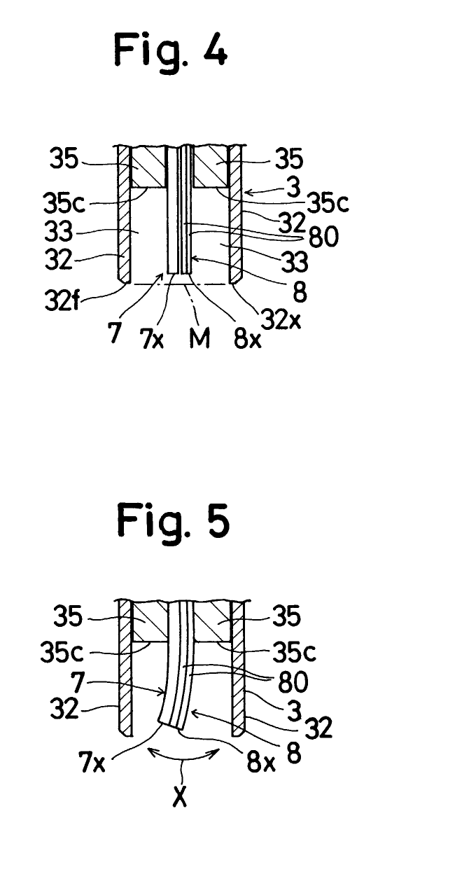

The fiber array 7 and the actuator 8 are accommodated in an inner chamber 33 of the probe 32 of the holder 3 and are lined by a fixing block member 35 which is formed of either synthetic resin or metal. It is to be noted that distal ends 7x and 8x of the fiber array 7 and actuator 8 are projected from an outer surface 35c of the fixing block member 35 so as to bend or to establish the snap action. Thus, as shown in FIG. 3, the lens 11 on the emitting optical fiber 1 and the wedge lenses 29 on the respective receiving optical fibers 21-27 are exposed on the surface 35a of the fixing block member 35. In addition, as shown in FIG. 4, the distal end 7x of the fiber array 7 is placed inside the chamber 33 by retracting from a phantom line M which is in line with a distal end surface 32f of the probe 32. It is to be noted that the position of the distal end 8x of the actuator 8 is made substantially identical with the position of the distal end 7x of the fiber array 7.

Upon voltage application to the actuator 8, as shown in FIG. 5, the distal end 8x of the actuator 8 which extends from the outer surface 35c of the fixing block member 35 is brought into a bending state or is made to do a snap action. Thus, the fiber array 7 which is next to the actuator 8 is also bent concurrently in a same direction. In this state, when the above voltage application is adjusted to change gradually, the bending degree of the actuator 8 also changes correspondingly, which makes it possible to change the bending degree of the fiber array 7. If the voltage to be applied to the actuator 8 increases drastically, correspondingly the degree of bending of the actuator 8 becomes much increased. In the present embodiment, either voltage increasing mode is possible. Upon interrupting the voltage application to the actuator 8, the actuator 8 is returned to its original shape, which causes the fiber array 7 to return to its original shape. It is to be noted that the actuator 8 formed of a piezoelectric element which is excellent in its response.

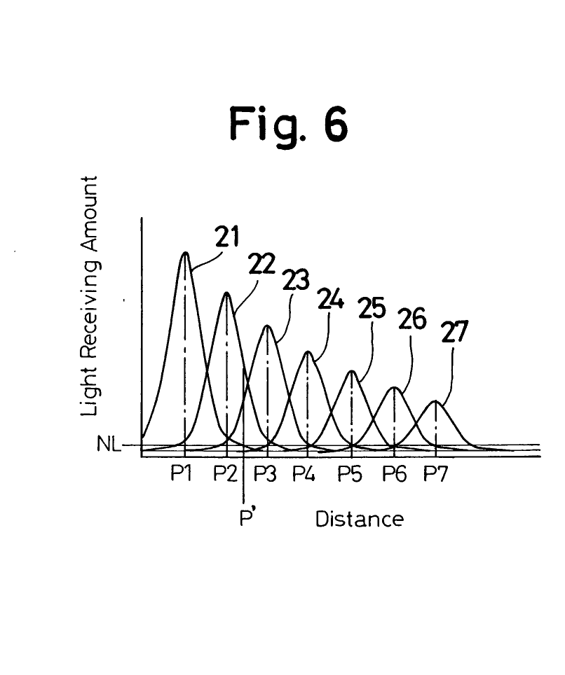

Next, with reference to FIGS. 6 and 7, the measuring principle used the periodontal pocket depth measuring device in accordance with the present embodiment will be described. As shown in FIG. 7, the distal end 11a of the lens 11 at the distal end of the emitting optical fiber 1 is made flat, so that the emitting optical axis 11r extends along the emitting optical fiber 1, while due to the inclined distal end 29a of the lens 29 of each of the receiving optical fibers 21-27, the optical axes 21r-27r thereof are made inclined relative to the optical axis 11r of the emitting optical fiber 1. Thus, the optical axes 21r-27r of the respective receiving optical fibers 21-27 are made inclined, as they extend downwardly, toward the optical axis 11a of the emitting optical fiber 1. It is to be noted that the optical axes 21r-27r of the respective receiving optical fibers 21-27 are closely related and are in high region.

As best shown in FIG. 7, the optical axes 21r, 22r, 23r, 24r, 25r, 26r, and 27r of the respective receiving optical fibers 21, 22, 23, 24, 25, 26, and 27 intersect the optical axis 11r of the emitting optical fiber 1 at positions 21p, 22p, 23p, 24p, 25p, 26p, and 27p which are at distances P1, P2, P3, P4, P5, P6, and P7, respectively, from the distal end surface 32f of the probe 32. As to the relationship between each distance, P1<P2<P3<P4<P5<P6<P7. The positions are designed to be spaced from one another in the light emitting direction.

An overlap area between the light emitting region of light emitting optical fiber 1 and the light receiving region of the light receiving optical fiber 21(22/23/24/25/26/27) attains a maximum at the point 21p(22p/23p/24p/25p/26p/27p). Depending on such an overlap area, the amount of light received at each of the receiving optical fibers 21, 22, 23, 24, 25, 26, and 27 varies.

As can be understood from the illustration of FIG. 7, the receiving optical fibers 21, 22, 23, 24, 25, 26, and 27 are arranged in side-by-side relationship fashion so as to make the points P1, P2, P3, P4, P5, P6, and P7 appear discretely along the optical axis 11r of the emitting optical fiber 11. For example, if the distance to the bottom of the periodontal pocket is found at the position P4, the overlap area between the light receiving region of the receiving optical fiber 24 and the light emitting region of the emitting fiber 1 is made maximum i.e., larger than the overlap area between the light receiving region of each of other receiving optical fibers 21, 22, 23, 25, 26, and 27 and the light emitting region of the emitting fiber 1. If the overlap area between the light receiving region of a specific receiving optical fiber and the light emitting region of the emitting fiber 1 is found to be maximum, the amount of the light received at the specific receiving optical fiber becomes maximum. It is to be noted that in FIG. 6 ‘NL’ denotes noise level. It is also to be noted that each of the points P1, P2, P3, P4, P6, P6, and P7 is indicative of the distance X to the bottom of the periodontal pocket 91. As the distance to the bottom of the periodontal pocket 91 increases, the receiving optical fiber which has the maximum amount of light received thereat changes in the order of the receiving optical fibers 21, 22, 23, 24, 25, 26, and 27.

In the present embodiment, there are two methods for calculating or determining the distance X between the distal end of the probe 32 and the bottom of the periodontal pocket 91.

A first method is as follows: The distances P1, P2, P3, P4, P5, P6, and P7 are corresponded to the respective receiving optical fibers 21, 22, 23, 24, 25, 26, and 27 in advance. Then, finding a receiving optical fiber whose receiving light amount is the maximum can determine the distance X between the distal end of the probe 32 and the bottom of the periodontal pocket.

A second method is as follows: A receiving optical fiber whose receiving light amount is the maximum and its neighboring receiving optical fiber are found. Then, a ratio of receiving light amounts among the receiving optical fibers is calculated. On the basis of such a ratio and the former receiving optical fiber, the distance X between the distal end of the probe 32 and the bottom of the periodontal packet is determined. For example, assuming that the amount of light received at the receiving optical fiber 22, its neighboring optical fiber 21(23) is found. Then, a ratio of receiving light amount between the receiving optical fibers 22 and 21(23) is found to determine the distance.

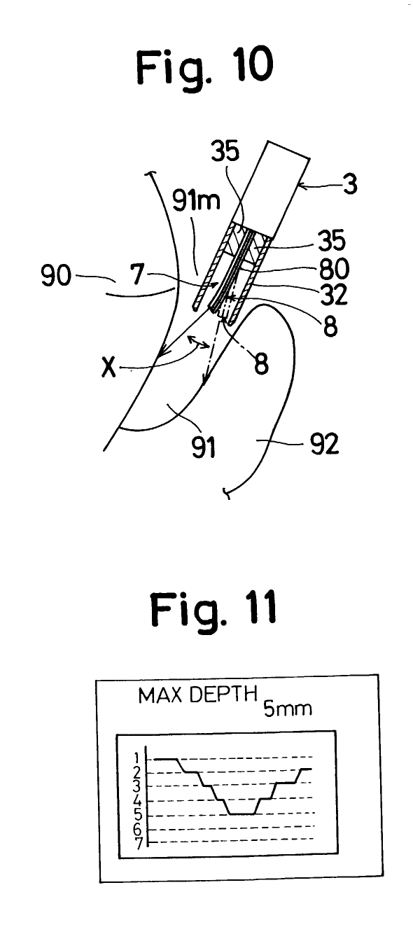

Measuring the distance X to the bottom of the periodontal pocket 91 of the teeth 90 will be explained with reference to FIGS. 8 to 12 inclusive. As shown in FIGS. 8 and 10, the probe 32 is placed at the top of the periodontal pocket 91 which is defined between the teeth 90 and the gum 92. At this time, a scale 32m marked on an outer surface of the distal end of the probe 32 is aligned with a distal end 92m of the gum 92 to define a reference point or an origin of measuring. After turning on the switch 66, the probe 32 is moved from a point A to a point B along a front side (alternately rear side) of the teeth 90 at a low speed. Then, the switch 66 is turned off. During such a movement of the probe 32 from the point A to the point B, as shown in FIG. 10, a scanning is made along a path between the teeth 90 and the gum 92 such that the distal end 7x of the fiber array 7 is made to bend (snap action) in the X-direction. A single bending movement of the distal end 7x of the fiber array 7 from the gum 92 toward the teeth 90 constitutes one cycle of scanning and vice versa. During the above movement of the probe 32 from the point A to the point B, the scanning (i.e., the bending movement or snap action) is made in plural cycles.

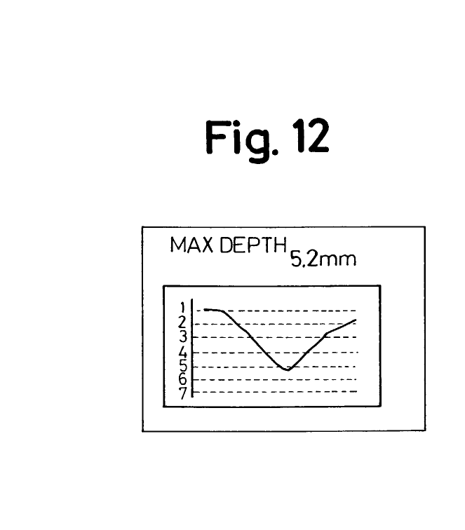

During the above scanning, the maximum distance (i.e. the depth) of the periodontal pocket 91 is determined per cycle to display on the data indication portion. FIGS. 11 and 12 illustrates the maximum distance (i.e. the depth) of the periodontal pocket 91 in time series in digital and analogue mode, respectively. In a graph in each of FIGS. 11 and 12, horizontal and vertical axes represent a distance between the points A and B and the depth of the periodontal pocket, respectively: The data readable from the vertical axes includes the depth found in the scanning. Thus, understanding the pocket bottom condition can be established by moving the probe 32 in the A-B direction and scanning in the X-direction, which is effective in dental treatment.

In the present embodiment, the actuator 8 is formed in a sheet configuration whose thickness direction extends along the width of the opening of the periodontal pocket 91, which enables the probe 32 to reduce its width DA. Thus, as shown in FIG. 8, even if the width of the opening or distal end 91m of the periodontal pocket 91 is narrow, no trouble can occur when the probe 32 its positioned opposed thereto.

In the present embodiment, though the distal end 7x of the fiber array 7 is exposed from the outer surface 35c of the fixing block member 35 for being brought into bending movement or snap action, the distal end 7x is held or supported by the sheet-like actuator 8, which makes the distal end 7x of fiber array 7 free from the possible idle movements while the device is inactive, resulting in the prevention of damage to the distal end 7x of the fiber array 7.

[Second Embodiment]

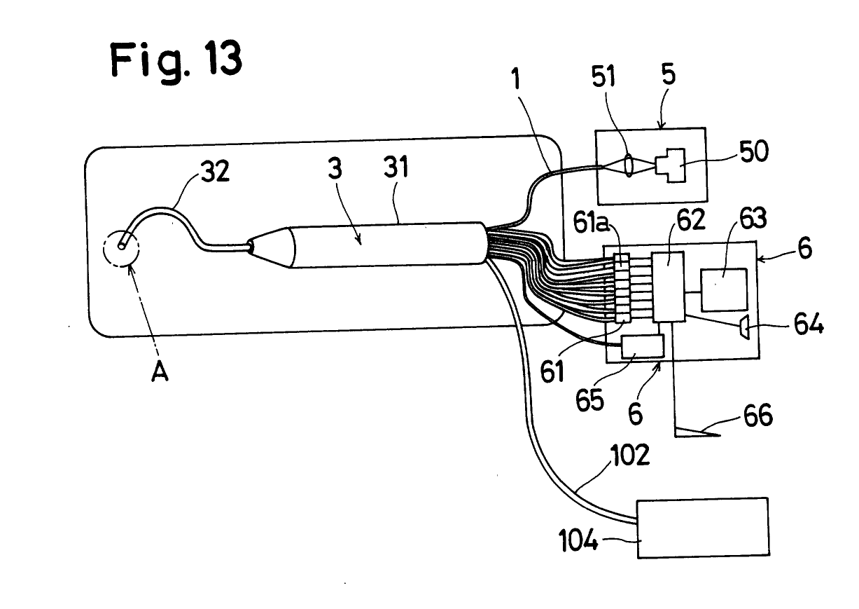

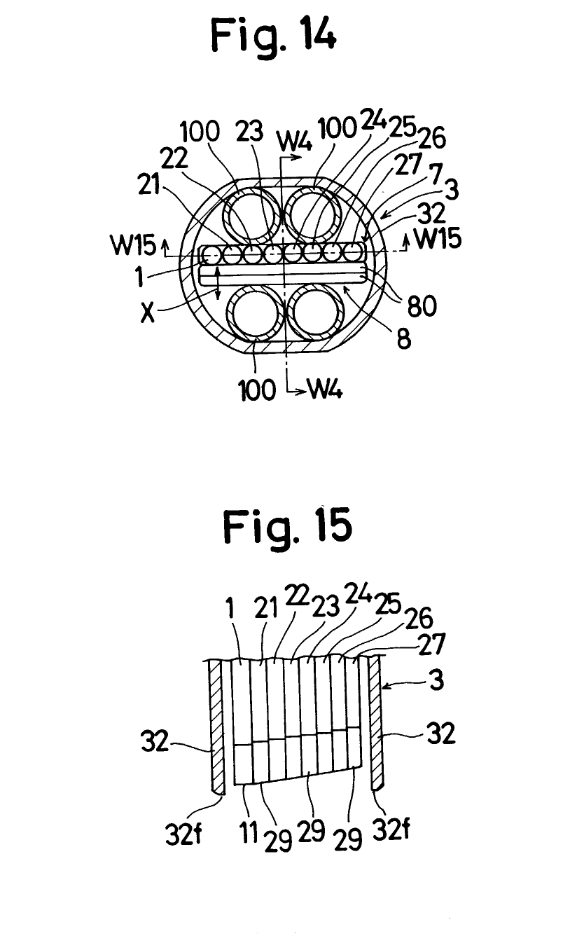

Referring first to FIGS. 13 to 15 inclusive, there is illustrated a periodontal pocket depth measuring device as an example of a distance measuring device in a second embodiment of the present invention. The second embodiment is identical with the first embodiment in construction except that the former has nozzles 100. The fiber array 7 and the actuator 8 am placed between a first set of two nozzles 100 and a second set of other two nozzles 100. The nozzles 100 eject fluid such as water or gas to expand the opening of the periodontal pocket 91 if the opening is closed or is too narrow to fit the probe 32.

[Third embodiment]

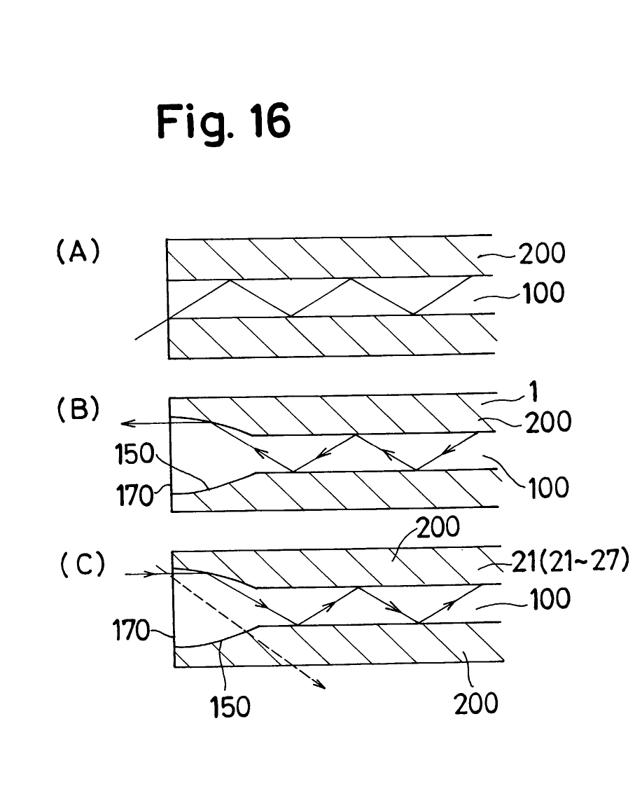

As shown in FIG. 16(A), the optical fiber is made up of a core 100 and a cladding 200 surrounding the core 100. In addition, the light beam which goes into the core 100 makes total reflections to travel through the core 100. As shown in FIG. 16(B), a core 100 of the emitting optical fiber 1 has an integral radially enlarged portion 150 at a distal end surface 170 for limiting the expansion of the light beam. As shown in FIG. 16(C), a core 100 of the receiving optical fiber 21(22/23/24/25/26/27) has an integral radially enlarged portion 150 at a distal end surface 170 for prevention the entrance of a disturbance light beam, which is indicated by phantom line, into the core 100.

The invention has thus been shown and description with reference to specific embodiments, however, it should be understood that the invention is in no way limited to the details of the illustrates structures but changes and modifications may be made without departing from the scope of the appended claims.

What is claimed is:

1. A distance measuring device comprising:
emitting means for emitting a light beam from a distal end thereof to an object so that the light beam may be reflected on the object;
receiving means for receiving the reflected beam at a distal end thereof;
a holder holding the emitting means and the receiving means;
an actuator which moves the distal ends of the respective emitting means and receiving means Concurrently and in a same direction; and
analyzing means for determining a distance from the emitting means to the object on the basis of a signal derived from the light beam received at the distal end of the receiving means.

2. A distance measuring device as set forth in claim 1, wherein the actuator is selected from one of a piezoelectric element, a bimetal element, a magentostrictor, and an electromagnetic actuator.

3. A distance measuring device as set forth in claim 1, wherein the receiving means comprises a plurality of side-by-side arranged optical fibers.

4. A distance measuring device as set forth in claim 3, wherein the actuator is selected from one of a piezoelectric element, a bimetal element, a magentostrictor, and an electromagnetic actuator.

5. A distance measuring device as set forth in claim 1, wherein the object is a bottom of a periodontal pocket and wherein the actuator has a sheet configuration whose thickness is directed to a teeth alignment direction.

6. A distance measuring method comprising the steps of:
emitting a light beam from an emitter to an object so that the light beam may be reflected on the object;
receiving the reflected beam at a receiver;
holding the emitter and the receiver;
moving distal ends of the respective emitter and receiver concurrently and in a same direction; and
determining a distance from the emitter to the object on the basis of a signal derived from the light beam received at the distal end of the receiver.

7. A distance measuring method as set forth in claim 6, wherein the object is a bottom of a periodontal pocket.
